(19) United States
(12) Patent Application Publication
(10) Pub. No.: US 2006/0275174 A1
(43) Pub. Date: Dec. 7, 2006

(54) Transportable measuring apparatus utilizing hydrogen flame

(75) Inventors: Kazuya Matsushita, Kyoto-shi (JP); Masahiko Endo, Kyoto-shi (JP); Aya Taniguchi, Kyoto-shi (JP); Satoshi Inoue, Kyoto-shi (JP); Hiroshi Mizutanii, Kyoto-shi (JP)

Correspondence Address: BROOKS KUSHMAN P.C., 1000 TOWN CENTER, TWENTY-SECOND FLOOR, SOUTHFIELD, MI, 48075, US

(73) Assignee: Horiba, Ltd., Kyoto-shi (JP)

(21) Appl. No.: 11/434,363

(22) Filed: May 12, 2006

(30) Foreign Application Priority Data
May 13, 2005 (JP) 2005-140795
Dec. 7, 2005 (JP) 2005-353323

(51) Int. Cl. G01N 33/00

(52) U.S. Cl. 422/54; 422/51

(57) ABSTRACT

There is provided a general-purpose transportable measuring apparatus utilizing a hydrogen flame capable of measurement for a long time by simple constitution. The transportable measuring apparatus includes: a hydrogen feeding means having Metal Hydride as fuel gas is used, a means of refining air as a burner air is used, and a battery is used as a means of feeding power to the apparatus.

### BACKGROUND OF THE INVENTION

1. Field of the Invention

The present invention relates to a transportable measuring apparatus utilizing a hydrogen flame, which is useful particularly as a transportable flame ionization measuring apparatus utilizing Metal Hydride as a hydrogen feeding means.

2. Description of the Related Art

Usually, an air pollution measuring apparatus such as an apparatus for measuring a generating source, an apparatus for measuring environmental air or an apparatus for measuring an automobile exhaust gas constitutes a sampling system provided with a filter, a switching valve, a sample introduction tube, a dehumidifier, a suction pump, a throttling valve and a flow meter between a sample collection position and an analyzer for the purpose of dehumidifying a sample fluid, removing dust or maintaining a constant flow rate as shown in FIG. 11, and the measuring apparatus is fixed and operated near to the sample collection position (see JIS B 7982-2002).

As a method of measuring non-combustion hydrocarbons or hydrocarbons generated upon incomplete combustion in the air or exhaust gas, flame ionization detection (hereinafter, an analyzer using flame ionization detection is referred to as “FID analyzer”) is frequently used. The FID analyzer is used widely because the amount of carbon (that is, a hydrocarbon concentration) ionized in a hydrogen flame is extracted and detected as a change in potential by an electrode arranged around the hydrogen flame, and a very small amount of components can be measured with high detection sensitivity, and highly accurate measurement excellent in linearity is feasible.

Similarly, there is a flame photometric detection measuring apparatus for measuring sulfur oxides in the air, by which the amount of the flame in a specific wavelength range (that is, the concentration of sulfur in a sample) generated by a hydrogen flame is detected by a light detector, and a very small amount of components can be measured with high detection sensitivity.

The measuring apparatus for continuously measuring the concentration of a specific component in a sample by a detector using a hydrogen flame requires a fuel gas (high-purity hydrogen) and a burner air (refined air) and uses a large and heavy high-pressure gas container to feed the gas, and is thus generally used as a stationary apparatus.

However, the conventional measuring apparatus using a hydrogen flame should solve the following problem.

That is, the conventional measuring apparatus requires a high-pressure gas container for the fuel gas and refined air, and when used as a transportable measuring apparatus, is limited to the one carried in vehicles, and the apparatus should be transferred to a previously specified place where it can be installed and the high-pressure gas containers are prepared as a separate unit, thus limiting measurement conditions to restrict applicability.

In such measuring apparatus, a high-pressure gas container of small volume can also be used for fuel gas and refined air, but when the volume is decreased, the measurable time is reduced due to the characteristics of conventional FID and FPD, and the intended measurement time cannot be secured in some times. On the other hand, when the flow rate of fuel gas or refined air is lowered under the current working conditions of FID and FPD, there arises a problem of failure to secure predetermined characteristics of FID and FPD, such as reduction in detection sensitivity or change in relative sensitivity of hydrocarbon.

The detection accuracy in the measuring apparatus using a hydrogen flame is influenced significantly by the state of the hydrogen flame in a detector, so regulation of the flow of fuel gas and burner air, that is, regulation of the pressure in feeding the gas to the analyzer requires very high accuracy. Specifically, the pressure of both the fuel gas and burner air should be regulated accurately at about 0.01 to 0.05 MPa. On the other hand, both the gases are sealed into a high-pressure gas container at 10 MPa or more so that when the gas is introduced at a reduced pressure of not more than 0.1 MPa into the analyzer, two-stage depressurization should inevitably be used as the mechanism of depressurization.

In measurement of a specific component such as hydrocarbon or sulfur oxide at a very low concentration in the environmental air, the influence of impurities in fuel gas and burner air cannot be negligible. For example, when a high-pressure gas container is used as a means of feeding hydrogen as the fuel gas, a purity of up to 99.999% is general, so the fuel gas used should be refined with a charcoal filter arranged in a fuel gas-feeding line, and the working of the measuring apparatus is checked separately with a calibration gas of known concentration.

Not only the stationary measuring apparatus but also the transportable measuring apparatus should be inevitably installed in a position apart from a sample collection site, thus requiring arrangement of a heating piping system and a large-volume suction pump. Accordingly, the apparatus should receive power from a general commercial power source or an instrumented power source, which is troublesome in constituting the transportable measuring apparatus.

### SUMMARY OF THE INVENTION

To solve the problem, the object of the present invention is to provide a general-purpose transportable measuring apparatus utilizing a hydrogen flame capable of measurement for a long time by simple constitution. Particularly, the object of the present invention is to provide a highly safe and simple transportable measuring apparatus utilizing a hydrogen flame by securing a fuel-gas source and a refined-air source not requiring high-pressure gases.

As a result of extensive study, the present inventors have found that the object can be achieved by the following transportable measuring apparatus using a hydrogen flame, and the present invention has been thereby completed. As used herein, the “transportable measuring apparatus” refers broadly to a movable measuring apparatus, and includes movable apparatuses used in stationary measurement for a long time.

That is, the present invention relates to a measuring apparatus for continuously measuring the concentration of a specific component in a sample by a detector utilizing a hydrogen flame, wherein a hydrogen feeding means having Metal Hydride as fuel gas is used, a means of refining air as a burner air is used, and a battery is used as a means of feeding power to the apparatus.

In the measuring apparatus utilizing a hydrogen flame, such as FID analyzer and FPD analyzer, the property of a specific substance such as hydrocarbon to exhibit an inherent reaction in a hydrogen flame is utilized to achieve highly selective measurement. To form a hydrogen flame, the fuel gas and burner air are required as described above. In the present invention, a low-pressure container (less than 1 MPa) having Metal Hydride sealed therein and a simple means of refining air are used as the means of feeding fuel gas and burner air, in place of the conventional high-pressure container (1 MPa or more), thus attaining constitution of a transportable measuring apparatus which is hardly arrived at in the prior art. Thus, high-pressure-related hazard can be eliminated, and safety can be improved.

As a means of feeding electric power to the apparatus, a battery can be used in place of the general power source thereby simplifying the constitution of the transportable apparatus and enabling measurement in a place nearer to a sample collection position, and by reduction in heating of a piping and in the capacity of a pump, the electric power used can be further reduced.

The hydrogen feed means having Metal Hydride can give stable supply of hydrogen of ultrahigh purity (99.999% or more) by the function of Metal Hydride to absorb hydrogen selectively. Accordingly, in the detector utilizing a hydrogen flame, a broad plateau region of hydrogen pressure capable of durable ignition by stable supply of hydrogen can be secured, and the flow of hydrogen fed can be reduced, that is, the fuel gas can be used for a long time. Such function to absorb hydrogen selectively provides refining function for fuel gas, and therefore, no separate refining means is necessary for a fuel gas line.

As described above, Metal Hydride is utilized by sealing it into a small and lightweight container, and as the burner air, air is easily refined in the analyzer, and the apparatus is driven by a battery as the power source, whereby the general-purpose transportable measuring apparatus utilizing a hydrogen flame capable of measurement for a long time by simple constitution can be provided.

The present invention relates to the transportable measuring apparatus utilizing a hydrogen flame, which comprises a single depressurization mechanism in a hydrogen gas line fed from the hydrogen feeding means to the detector.

As described above, two-stage depressurization should be inevitably utilized in the prior art as the mechanism of depressurization from a pressure of 10 MPa or more. In the present invention, one-stage depressurization can be realized by using a small container into which Metal Hydride having a low pressure of less than 1 MPa is sealed. In addition, one-stage depressurization can be realized even in a burner air line by using a simple air refining means and a pump as a means of feeding the burner air. This depressurization mechanism can give rise to improvement in the accuracy in regulation of pressure and simplification of the flow channel, thus contributing to downsizing of the apparatus.

Because of a charging pressure of less than 1 MPa, re-charging can be easily carried out by the general user, and the running cost required for high-pressure gas container can be reduced, the labor involved in taking-in or taking-out the high-pressure gas can be reduced, and the appointed period of from manufacture to delivery of high-pressure gas can be reduced.

The present invention relates to the transportable measuring apparatus utilizing a hydrogen flame, wherein Metal Hydride is composed fundamentally of an AB5-type Metal Hydride.

A hydrogen feeding means working under ambient-temperature conditions is preferable for constitution of the measuring apparatus. In the present invention, Metal Hydride composed fundamentally of an AB5-type Metal Hydride that is a metal-bound-type hydride having a standard decomposition temperature of about 50° C. or less can be utilized to allow dehydrogenation reaction to proceed at almost ordinary temperatures without necessity for heating the hydrogen feeding means at high temperatures. Accordingly, the simplification and energy-saving design of the measuring apparatus are feasible without specifying that the analyzer and piping system are durable to high temperatures. In addition, the present invention contributes to realization of the transportable measuring apparatus driven by a battery. As used herein, the AB5-type Metal Hydride refers to Metal Hydride (LaNi₅, MmNi₅ etc.) composed fundamentally of a lanthanum-nickel-type (La—Ni-type) alloy, which will be described later in more detail.

The present invention relates to the transportable measuring apparatus utilizing a hydrogen flame, wherein the top of a nozzle for forming a hydrogen flame in the detector is flat, and the diameter of the flow channel in a fuel gas-jetting part at the top of the nozzle is smaller than the diameter of the flow channel in the inside of the nozzle.

For realizing the transportable measuring apparatus utilizing a hydrogen flame, it is necessary to secure a small and lightweight means of feeding a large-volume gas and reduce the amount of fuel gas and burner air consumed in the analyzer. The present inventors extensively studied the top of a nozzle in forming a hydrogen flame in the detector, and as a result, they found that the shape of the top of the nozzle is flat, and the diameter of the flow channel in a fuel gas-jetting part at the top of the nozzle is smaller than the diameter of the flow channel in the inside of the nozzle, whereby even if the flow rate of fuel gas and burner air fed is lowered, the detection sensitivity can be secured and the predetermined characteristics of FID and FPD, such as relative sensitivity of hydrocarbon, can be secured. It was thus possible to provide a general-purpose transportable measuring apparatus utilizing a hydrogen flame capable of measurement for a long time by simple constitution.

The present invention relates to the transportable measuring apparatus utilizing a hydrogen flame, wherein impurities contained in the hydrogen feeding means are detected within a warm-up time of the apparatus thereby checking the working of the detector.

As described above, hydrogen occluded in Metal Hydride can be fed stably with ultrahigh purity at low pressure, but the apparatus including component parts or a piping system constituting the hydrogen feeding means cannot be said to be completely free of impurities, and according to the inventors' finding, it was revealed that particularly just after supply of hydrogen is initiated, hydrocarbons or sulfur compounds are contained in a very small amount in the ppb order or in a level of 1 ppm or less, and after a predetermined time, these impurities are reduced to a hardly detectable level. In the present invention, this phenomenon can be utilized to make it possible to judge whether the detector normally works within the warm-up time, by detecting a change in the background due to hydrocarbons contained as impurities during the predetermined time after ignition of hydrogen without passing a sample gas or a calibration gas. By initiating measurement with the background in a stabilized state, it is not necessary to arrange an impurity eliminating filter (for example, a charcoal filter) essential where high-pressure gas is used in the prior art.

As described above, the present invention can be applied to provide a general-purpose measuring apparatus utilizing a hydrogen flame by simple constitution. In addition, fuel gas and burner air are fed at low pressures thereby achieving the safety of the measuring apparatus or improving the accuracy in regulation of pressure and reducing the electric power used and downsizing the apparatus.

### BRIEF DESCRIPTION OF THE DRAWINGS

FIG. 1 is a view for illustrating a fundamental constitution (first constitutional example) of the measuring apparatus according to the present invention;

FIG. 2 is a view for illustrating another constitution (second constitutional example) of the measuring apparatus according to the present invention;

FIG. 3 is a view for schematically illustrating the structure of a detector in an FID analyzer installed in the measuring apparatus according to the present invention;

FIG. 4 is a view for schematically illustrating the structure of the top of a detector nozzle in the FID analyzer;

FIG. 5 is a view for illustrating the detection sensitivity and fuel gas flow characteristics of a detector in the FID analyzer;

FIG. 6 is a view for illustrating the oxygen interference characteristics of a detector in the FID analyzer;

FIG. 7 is a view for illustrating the relative sensitivity characteristics of hydrocarbon in a detector in the FID analyzer;

FIG. 8 is a view for illustrating the flow rate characteristics of Metal Hydride according to the present invention;

FIG. 9 is a view for illustrating the integrated flow rate characteristics of Metal Hydride according to the present invention;

FIG. 10 is a view for illustrating a method of using a hydrogen feeding means; and

FIG. 11 is a view for illustrating the constitution of a measuring apparatus according to the prior art.

### DETAILED DESCRIPTION OF THE INVENTION

Hereinafter, the present invention is described in detail by reference to the drawings.

### FUNDAMENTAL CONSTITUTION OF THE TRANSPORTABLE MEASURING APPARATUS UTILIZING A HYDROGEN FLAME ACCORDING TO THE INVENTION (FIRST CONSTITUTIONAL EXAMPLE)

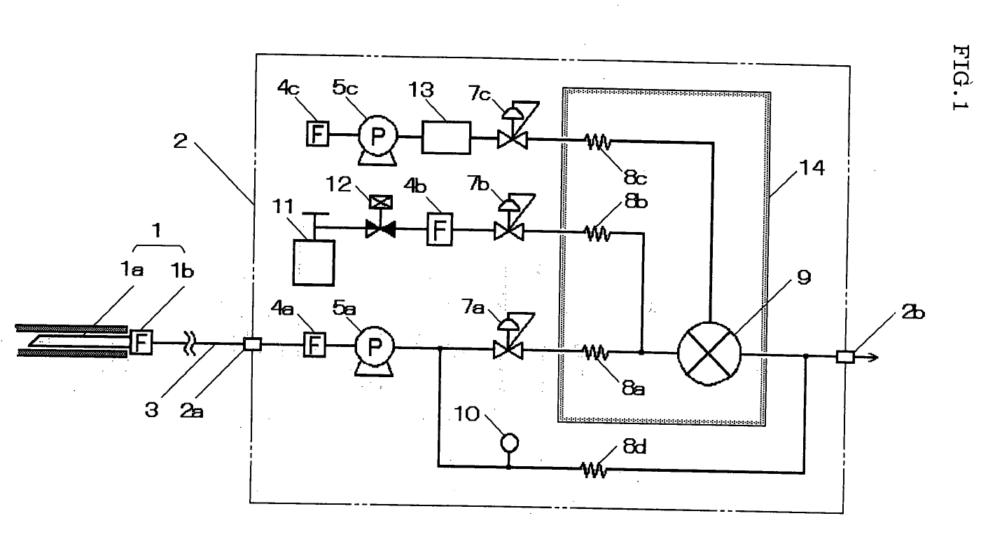

FIG. 1 shows a transportable apparatus for measuring hydrocarbon in automobile exhaust gas, including an FID analyzer, as one embodiment of the apparatus in the present invention. Specifically, the apparatus comprises a sample collection part 1 consisting of a sample collection tube 1a and a primary filter 1b, a transportable measuring apparatus 2 including an FID analyzer, and a sample introduction tube 3 connecting the two. As electric power fed to the transportable measuring apparatus 2, a build-in battery (not shown in the figure) is utilized.

A sample suctioned from the sample collection part 1 via a sample gas inlet 2a and secondary filter 4a by pump 5a is regulated at constant pressure by a pressure regulator 7a, allowed to flow at constant flow rate by a throttling valve 8a, and introduced into an FID analyzer 9. A part of the sample is combined with exhaust gas via flow rate sensor 10 and throttling valve 8d from the FID analyzer 9, and discharged from an outlet 2b. The flow rate of the sample collected varies depending on the required specifications of response speed in measurement, but is usually about 1 L/min., and a sample is introduced at about 10 to 100 mL/min. into the FID analyzer 9.

The fuel gas in the FID analyzer 9 is fed from a hydrogen feeding means 11 having Metal Hydride, is regulated at constant pressure via a stop valve 12 and filter 4b by a pressure regulator 7b, allowed to flow at constant flow rate by a throttling valve 8b, mixed with a sample from the throttling valve 8a, and introduced into the FID analyzer 9. The stop valve 12 is actuated at the time of working of the FID analyzer 9, whereby the fuel gas can be introduced at a predetermined flow rate into the FID analyzer 9. The filter 4b is arranged to remove impurities contained in hydrogen fed from the hydrogen feeding means 11, and preferably uses granular activated carbon, a charcoal filter or molecular sieves to remove residual materials at the time of production of Metal Hydride, or a very small amount of hydrocarbons and sulfur compounds generated from the surface of the member constituting the hydrogen feeding means 11.

By utilizing the hydrogen feeding means 11 having Metal Hydride, the fuel gas can be fed at a low pressure of less than 1 MPa, and supply of the fuel gas into the FID analyzer 9 by one-stage depressurization to 0.01 to 0.1 MPa can be realized. By utilizing a small container which can feed the same amount of hydrogen, though having a volume of about ½ to 1/10 relative to the volume of a conventional high-pressure gas container, the measuring apparatus can be downsized as a whole.

The flow rate of the fuel gas significantly influences the characteristics of the detector, such as detection sensitivity, oxygen interference characteristics and relative sensitivity of hydrocarbon, and thus varies depending on specifications such as gases to be measured and coexisting components in a sample, but usually the fuel gas is introduced at about 10 to 100 mL/min. into the FID analyzer 9. The flow rate is regulated between the flow rate of a sample and the flow rate of burner air. The transportable measuring apparatus enables measurement for a long time by minimizing the flow rate of the fuel gas while securing predetermined characteristics of the detector.

As used herein, oxygen interference characteristics refer to a change in sensitivity resulting from the change in the state of hydrogen flame caused by the concentration of oxygen in a sample. The relative sensitivity of hydrocarbon means that although the output from the detector in the FID analyzer is ideally proportional to the number of carbon atoms, the output from the detector varies according to a change in hydrogen flame or a difference in the process of ionization in actual measurement. As described later, for example, the output of methane (CH₄):output of acetylene (C₂H₂) is not 1:2, and the output of C₂H₂ and the output of ethylene (C₂H₄) or ethane (C₂H₆) are not to equal to each other at the same concentration.

The burner air in the FID analyzer 9 is suctioned via filter 4c by pump 5c to remove hydrocarbons mainly by an air refining means 13, and then regulated at constant pressure by a pressure regulator 7c, allowed to flow at constant flow by a throttling valve 8c, and introduced into the FID analyzer 9. The flow rate of the burner air is varied depending on specifications of coexisting components and gases to be measured, but usually the burner air is introduced to the FID analyzer 9 at about 100 to 500 mL/min. which is several times as high as the flow rate of gas completely combusted.

The air refining means 13 makes use of a method that involves oxidizing hydrocarbons into carbon dioxide and water and removing the water, or a method that involves removing hydrocarbons etc. by adsorption into an adsorbent and a water eliminating agent. The latter is preferable for reducing electric power for the transportable measuring apparatus, and it is preferable to use silica gel, activated alumina or molecular sieves as the water eliminating agent and granular activated carbon, a charcoal filter or molecular sieves as the adsorbent.

The throttling valves 8a, 8b and 8c as the final flow rate-regulating means in the sample line, the fuel gas line and the burner air line into the FID analyzer 9 are designed so as not to undergo a change in ambient temperature, and together with the FID analyzer 9, are regulated preferably at a predetermined temperature (for example 50 to 60° C.), and FIG. 1 shows arrangement thereof on a thermostatic block 14. The FID analyzer 9 and the hydrogen feeding means 11 will be described later in detail.

As shown in FIG. 1, the transportable (portable) measuring apparatus can be constituted by using the hydrogen feeding mechanism utilizing a small and lightweight container having Metal Hydride in it and the burner air feeding mechanism using an air refining means with a battery as the power source to drive the apparatus.

### OTHER CONSTITUTIONAL EXAMPLE OF THE TRANSPORTABLE MEASURING APPARATUS UTILIZING A HYDROGEN FLAME ACCORDING TO THE PRESENT INVENTION (SECOND CONSTITUTIONAL EXAMPLE)

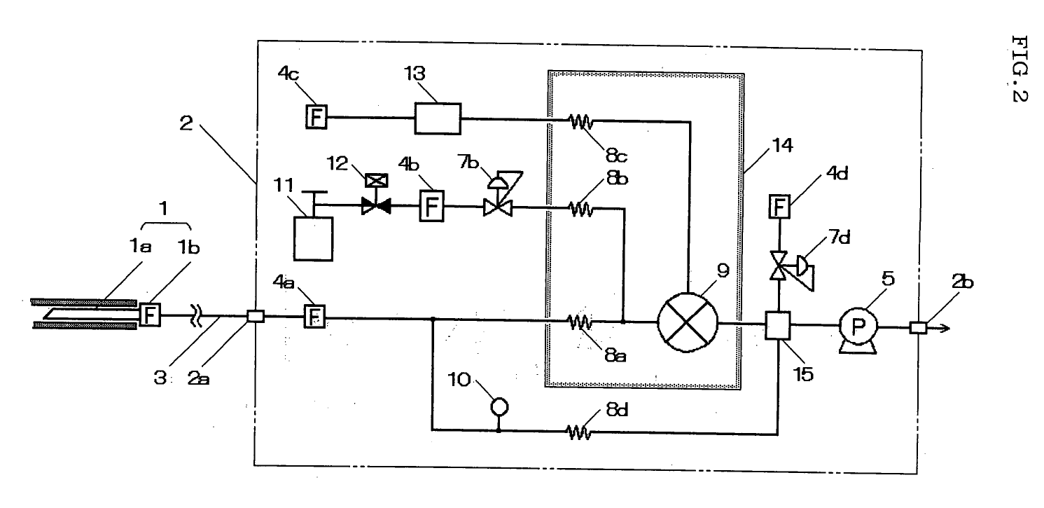

FIG. 2 shows the transportable measuring apparatus for measuring hydrocarbon in automobile exhaust gas, including a vacuum-type FID analyzer. Specifically, the measuring apparatus is provided with a depressurizing pump 5 via a buffer tank 15 after the FID analyzer 9, and the FID analyzer 9 is regulated at a predetermined pressure (for example, −0.005 MPa etc.) by a backpressure regulator 7d connected to the buffer tank 15. The backpressure regulator 7d can maintain a predetermined pressure by suctioning air via filter 4d.

By applying this constitutional example, one pump 5 can be used in place of the pumps 5a and 5c in FIG. 1. Further, one backpressure regulator 7d can be used in place of the pressure regulators 7a and 7c. Although filter 4d and buffer tank 15 are additionally used in this example, the number of parts in total can be reduced, and the apparatus can be further downsized.

### CONSTITUTIONAL EXAMPLE OF A DETECTOR IN THE FID ANALYZER

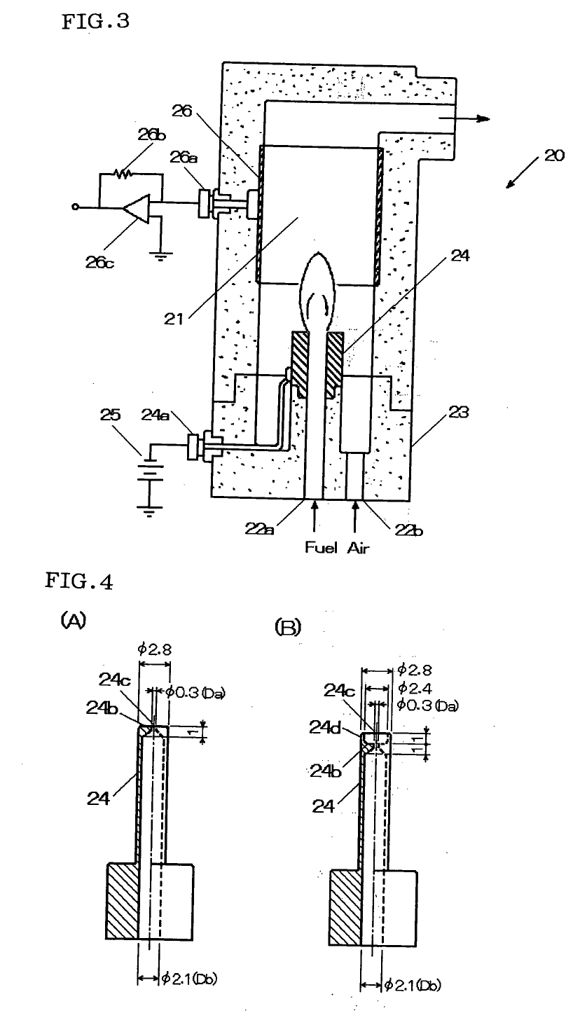

In a combustion chamber 21 in a detector 20 having e.g. the structure shown in FIG. 3 in the FID analyzer 9, the fuel gas (which becomes a gas having a sample mixed with hydrogen when the sample is detected) is fed through an inlet 22a, and air as the burner air is fed through an inlet 22b, whereby a hydrogen flame in the form shown in the figure is formed from the top of a stainless steel nozzle 24 formed integrally with a highly insulating block 23. The nozzle 24 is connected via lead 24a to a high-voltage power source 25, and a conductor 26 (made of, for example, a gold-plating layer) is arranged in the inside of the combustion chamber 21 of the block 23, and connected via lead 26a, high-resistance element 26b and amplifier 26c to a voltage recorder (not shown in the figure).

When high voltage is applied to the nozzle 24, ionized carbon is generated from a hydrocarbon component present in a sample. As a result, the potential of the conductor 26 is changed and inputted as a signal of concentration amplified by the amplifier 26c into the voltage recorder. The amount of ionized carbon is proportional with the number of carbons in the hydrocarbon component in the sample, and thus the potential of the conductor 26 is changed in proportion with the amount of the hydrocarbon component in the sample and can be recorded on the voltage recorder as a signal of the concentration of the hydrocarbon component in the sample.

[Characteristics Test on the Shape of the Top of the Detector Nozzle]

As shown in FIG. 4(A), it is preferable that the top 24b of the nozzle 24 is flat, and the diameter Da of the flow channel in the fuel gas jetting part 24c at the top 24b of the nozzle is smaller than the diameter Db of the flow channel in the inside of the nozzle 24 (nozzle A). By this constitution, approximately the same characteristics could be found to be attainable even if the flow rate of the fuel gas and the flow rate of the burner air are established to be lower than in a conventional gas-jetting nozzle having the diameter Db in the inside of the nozzle 24 illustrated in FIG. 3. That is, it was found that even if the flow rates of the fuel gas and burner air fed are lowered, the detection sensitivity can be secured, and the predetermined characteristics of FID and FPD, such as relative sensitivity of hydrocarbon, can be secured. It was thus possible to provide a general-purpose transportable measuring apparatus utilizing a hydrogen flame capable of measurement for a long time by simple constitution.

A further comparative test on the shape of the top was conducted in which the characteristics of the nozzle A were compared with those of the nozzle (nozzle B) wherein the top 24b of the nozzle 24 is flat, and the diameter Da of the flow channel in the fuel gas jetting part 24c at the top 24b of the nozzle is smaller than the diameter of Db of the flow channel in the inside of the nozzle 24, and simultaneously a protrusion 24d is arranged on the outer periphery of the nozzle, as shown in FIG. 4(B).

(1) Detection Sensitivity and Fuel-Gas Flow Rate Characteristics

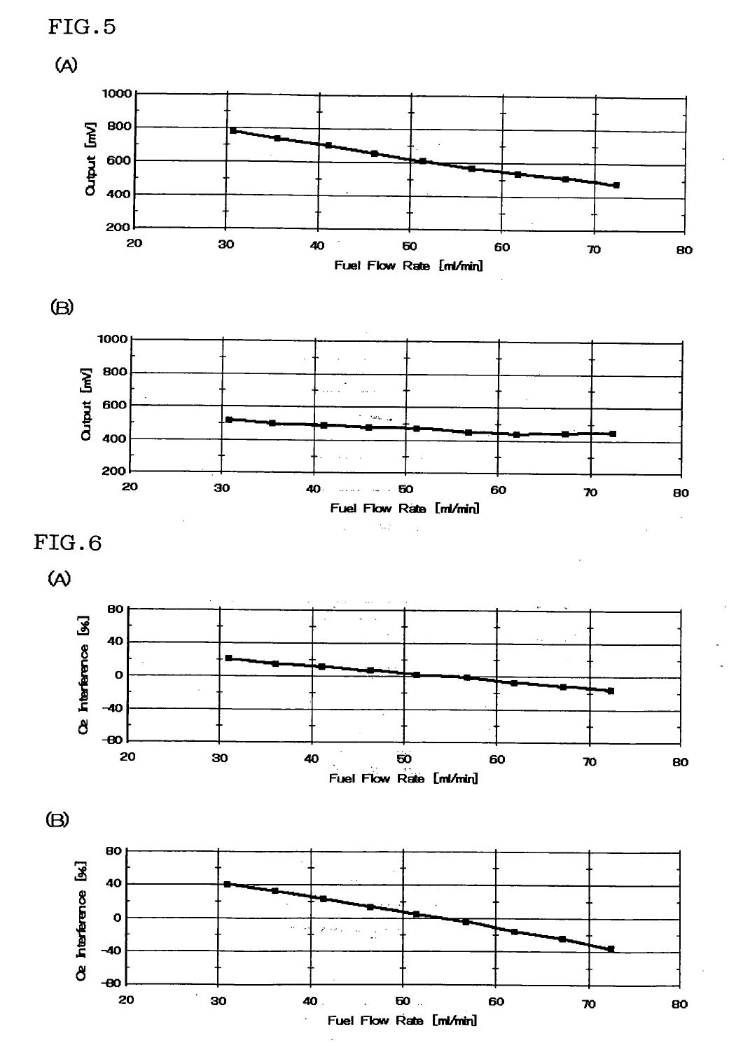

Detection sensitivity and fuel-gas flow rate characteristics were determined when the flow rate of the fuel gas through the nozzles A and B was changed in the range of about 30 to 70 mL/min. relative to the standard condition set at about 50 mL/min. The results are shown in FIGS. 5(A) and 5(B).

The detection sensitivity was higher in about 20% in the nozzle A, and higher stability in flow rate characteristics in this range was showed in the nozzle B. With respect to the nozzle A, in a further lower flow rate region, an increase in detection sensitivity and the presence of a stable region (plateau region) can be estimated.

(2) Oxygen Interference Characteristics

A change in detection sensitivity, that is, oxygen interference characteristics, were determined when the concentration of oxygen in a sample was changed to 0% (based on nitrogen gas) and 21% (based on air) and the fuel flow rate through the nozzles A and B was changed in the range of about 30 to 70 mL/min. relative to the standard condition set at about 50 mL/min. The results are shown in FIGS. 6(A) and 6(B).

In the range of the changed flow rate of the fuel gas, the change in detection sensitivity in the nozzle A is within about ±20%. On the other hand, the change in detection sensitivity in the nozzle B is within about ±40%, in the range of the changed flow rate of the fuel gas, to exhibit the significant influence of the interference.

(3) Relative Sensitivity of Hydrocarbon

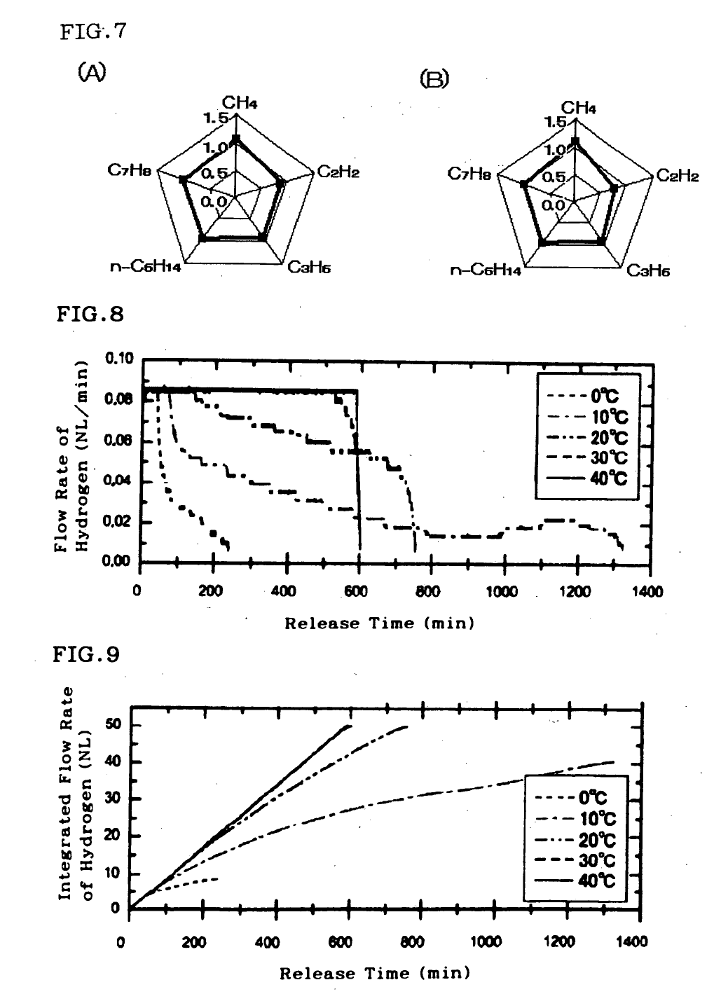

With propane (C₃H₈) as standard hydrocarbon, CH₄, C₂H₂, propylene (C₃H₆), n-hexane (n-C₆H₁₄) and toluene (C₇H₈) were measured, and the relative sensitivity thereof was determined from detection sensitivity per one carbon atom. The results are shown in FIGS. 7(A) and 7(B).

With respect to the nozzle A, the relative sensitivity is within the range of 0.9 to 1.1 relative to C₃H₈=1. With respect to the nozzle B, on the other hand, the relative sensitivity varies more significantly for C₂H₂ and CH₄.

(4) Summary

As described above, the shape of the top 24b of the nozzle 24 in the detector exerts an influence on the properties of the FID analyzer 9. It was found that when it is intended to achieve a reduction in the amount of consumed fuel gas which is one of the required characteristics of the measuring apparatus of the present invention, the shape of the nozzle A is preferable. As described above, the presence of a stable region (plateau region) can be estimated in the low flow-rate range, so the measuring apparatus though having a significantly high effect on reduction of the flow rate of fuel gas can be used even at a low flow rate of up to 30 mL/min. which is hardly attained in the prior art. Because various hydrocarbons can occur in combustion exhaust gas such as automobile exhaust gas, the shape of the nozzle A excellent in relative sensitivity is preferable for measurement of such exhaust gas. In measurement of hydrocarbons in the air, on the other hand, the background is stabilized, oxygen interference can be negligible, and the shape of nozzle B having a broad plateau region in the flow rate of the fuel gas is preferable where the main component is known to be CH₄.

[Outline of the Hydrogen Feeding Means]

Metal Hydride constituting the hydrogen feeding means 11 refers to an alloy exhibiting a reversible reaction in which the alloy upon contacting with hydrogen absorbs the hydrogen generating of heat, while the alloy upon heating releases the hydrogen, and specific examples include alloys such as titanium-iron-type, La—Ni-type, or magnesium-nickel-type. Comparison of Metal Hydrides with metal hydrides such as metal-bound-type hydride, covalent-bound-type hydride and ionic-bound-type hydride formed and introduced into a high-pressure gas container indicates that some have an ability to accommodate hydrogen in 6- to 7-fold density. Accordingly, Metal Hydride is a very effective means for the measuring apparatus of the present invention intended to reduce the size and weight of the hydrogen feeding means 11. In addition to the property of the alloy to occlude hydrogen in high density, Metal Hydride has various excellent properties as shown below, and in the present invention, these properties are effectively utilized to realize excellent functions.

Typical compositions of Metal Hydride are shown in Table 1.

TABLE 1

| Type | Metal Hydride |
|---|---|
| AB5 | LaNi₅ |
| | MmNi₅ |
| | CaNi₅ |
| AB2 | TiMn_{1.5} |
| | TiCr_{1.8} |
| | ZrMn₂ |
| | ZrV₂ |
| AB | TiFe |
| A2B | Mg₂Ni |
| Solid solution | Ti—Cr—V |

Particularly in examination results in the present invention, a fuel feeding means which is very superior in stable supply of hydrogen at room temperature can be secured by using an AB5-type Metal Hydride. The AB5-type Metal Hydride is based on a rare earth element, niobium, zirconium or Misch metal Mm (pyrophoric metal: an alloy of rare earth metals, an alloy of a rare earth metal and another element, a Zn—Sn-type or U—Fe-type alloy) as A and a catalytically active transition element (such as Al, Co, Cr, Fe, Mn, Ni, Ti, V, Zn or Zr) as B where the ratio of B to A is 5, and examples include LaNiS5 MmNi5 and CaNi5 shown in Table 1.

(1) Hydrogen can be Released Stably with Ultrahigh Purity.

Metal Hydride reacts selectively with hydrogen thereby forming a high-purity metal hydride and simultaneously occluding high-purity starting hydrogen, thus enabling stable release of ultrahigh (99.999% or more) hydrogen. Accordingly, when Metal Hydride is used as fuel gas in the FID analyzer 9, consumption of hydrogen at a low flow rate of up to 30 ml/min. can be realized. By the function of the alloy to absorb hydrogen selectively, ultrahigh-purity hydrogen can be obtained, and thus the arrangement of a refining means such as charcoal filter in a fuel gas line is not necessary. Specifically, impurities contained in a very small amount just after initiation of supply of the fuel gas are reduced to an undetectable level after the warm-up time of the measuring apparatus as shown in Table 2, so the measuring apparatus can also be constituted without arranging the filter 4b shown in FIG. 1 or 2.

TABLE 2

| | First | First | Second | Third | |
|---|---|---|---|---|---|
| | release | release | release | release | |
| | initial | after 6% | initial | Initial | Analysis |
| Composition | stage | release(*) | stage | stage | means(**) |
| H₂ [%] | 99.99913 | 99.99988 | 99.99990 | 99.99993 | — |
| O₂ [ppm] | <0.1 | <0.1 | <0.1 | <0.1 | GC-MS |
| N₂ [ppm] | 3.4 | 0.8 | 0.4 | 0.2 | GC-MS |
| CH₄ [ppm] | 4.9 | <0.1 | 0.2 | 0.1 | GC (FID) |
| HC [ppm] | <0.1 | <0.1 | <0.1 | <0.1 | GC (FID) |
| CO [ppm] | <0.1 | <0.1 | <0.1 | <0.1 | GC (FID) |
| CO₂ [ppm] | <0.1 | <0.1 | <0.1 | <0.1 | GC-MS |

(*)The gas after releasing 6% of the amount of occluded hydrogen was analyzed.
(**)GC is gas chromatography, and MS is mass spectrometry.
(2) The gaseous phase pressure in an occluded state can be reduced.

When the dissociation pressure in the operation temperature is about 0.2 to 0.5 MPa, and the release temperature is approximately constant, the pressure of hydrogen released from Metal Hydride can be stabilized. That is, a low-pressure container (less than 1 MPa) can be used to eliminate high-pressure-related hazard and improve safety. When a high-pressure container (1 MPa or more) is used, two-stage depressurization should be inevitably used as a depressurization mechanism in introducing a gas into the analyzer, while when a small container charged with Metal Hydride of low pressure less than 1 MPa is used, one-stage depressurization can be realized. Further, the charging pressure is less than 1 MPa, so re-charging can be easily carried out by the general user, and the running cost required for high-pressure gas container can be reduced, the labor involved in taking-in or taking-out the high-pressure gas can be relieved, and the appointed period of from manufacture to delivery of high-pressure gas can be reduced.

(3) The Procedure of Feeding Hydrogen is Feasible at Low (Room) Temperature.

If a procedure at high temperatures is necessary for constituting the transportable measuring apparatus, an increase in the capacity of a power source and additional use of a component part for heating the hydrogen feeding means and keeping it at high temperatures are required, which will be an obstacle to downsizing. In the present invention, it is preferable to use Metal Hydride composed fundamentally of an AB5-type Metal Hydride that is a metal-bound-type hydride having a standard decomposition temperature of about 50° C. or less. It is made thereby unnecessary to heat the hydrogen feeding means at high temperatures, and the dehydrogenation reaction can occur at almost ordinary temperatures. Accordingly, simplification of the measuring apparatus and design for energy saving are made feasible without specifying that the analyzer and piping system are durable to high temperatures.

(4) Initial Activity is Easy and Rapid Occlusion and Release are Feasible.

Initial activity means that hydrogen is occluded for the first time onto a metal, and Metal Hydride has high activity on occlusion and can release occluded hydrogen rapidly with temperature as an operation element. Because the characteristics of Metal Hydride can be effectively used in the reversible reaction of occluding and releasing hydrogen several times, the alloy has high applicability as resource and can reduce running cost. A low difference in equilibrium hydrogen-pressure (hysteresis) between occlusion and release in Metal Hydride can also be said to be an excellent aspect for operation in reversible re-use. Further, Metal Hydride is an alloy based on the metal, and thus has excellent heat conductivity and can be easily heated or cooled.

(5) The Width of the Plateau Region is Broad with Low Inclination.

Metal Hydride rapidly releases occluded hydrogen with temperature as an operation element, while the rate of release of hydrogen is extremely stabilized by stabilizing the operation temperature, as described below. That is, when Metal Hydride is regarded as a hydrogen feeding means, it has excellent characteristics such as broad plateau region and less change in the region with respect to the amount of hydrogen fed. By utilizing such characteristics as a source of feeding the fuel gas to the FID analyzer, a broad plateau region of hydrogen pressure capable of durable ignition can be secured, ultrahigh-purity (99.999% or more) hydrogen can be fed at a constant pressure, and the measuring apparatus utilizing a highly stable hydrogen flame can be formed.

(6) Metal Hydride is Resistant to Poison.

Metal Hydride is highly resistant to poisoning with impurities such as oxygen, carbon monoxide and water and has excellent corrosion resistance. That is, Metal Hydride is preferably applied to a transportable measuring apparatus requiring no particular treatment of the hydrogen feeding means even when not used, and requiring working conditions to be secured rapidly after transportation.

By utilizing the advantages described above, Metal Hydride composed fundamentally of an AB5-type Metal Hydride that is a metal-bound-type hydride having a standard decomposition temperature of about 50° C. or less is preferably used as a source of feeding the fuel gas to the transportable measuring apparatus utilizing a hydrogen flame as the object of the invention. Dehydrogenation reaction is feasible at almost ordinary temperatures without necessity for heating the hydrogen feeding means at high temperatures.

[Property Test of Metal Hydride]

The hydrogen release properties of Metal Hydride tank (MHSC-50L manufactured by The Japan Steel Works LTD.; referred to hereinafter as “MH tank”) were confirmed at a varying ambient temperature of the MH tank.

(1) Test Apparatus

The MH tank is arranged in an accommodating container and the ambient temperature of the accommodating container is changed. The pressure of hydrogen released from the MH tank is regulated by a pressure regulator, is sent to a capillary, and after regulation of flow rate, is measured for its flow rate (regulation of flow rate is not conducted when the flow rate is not higher than 0.1 L/min.).

(2) Contents of the Test

(2-1) Test Conditions

A release test was carried out under the 5 kinds of test conditions shown in Table 3.

TABLE 3

| | MH tank ambient | Hydrogen - introduction |
|---|---|---|
| No. | temperature | temperature |
| 1 | 0° C. | 20° C. |
| 2 | 10° C. | |
| 3 | 20° C. | |
| 4 | 40° C. | |

(2-2) Test Procedures
a. The MH tank is arranged in a water bath kept at 20° C., and then charged with hydrogen by introducing hydrogen at 1 MPa.
b. The charged MH tank is transferred to an incubator having a predetermined internal temperature and connected to a release piping.
c. After the MH tank is left for about 1 hour to attain a predetermined temperature, a valve is opened to initiate release.
d. When the flow rate of hydrogen becomes almost 0, the release test is finished.

(3) Test Results

The flow rate characteristics at each release temperature are shown in FIG. 8, and the integrated flow rate characteristics are shown in FIG. 9. In the stationary region, hydrogen was released at a flow rate of 0.085 L/min. As the ambient temperature is increased, the stationary release time tends to be increased. Because the flow rate curves at 20° C. and −40° C. and the integrated flow rate curves almost overlap with each other, and thus when the ambient temperature of the MH tank is 20° C. or more, hydrogen can be released at the constant flow rate until the tank is almost emptied. At 10° C., the flow rate was initiated to drop after 2 hours, but the flow rate of 0.05 L/min. was kept over 6 hours or more. At 0° C., however, the pressure dropped to less than 0.05 L/min. before the end of release of hydrogen.

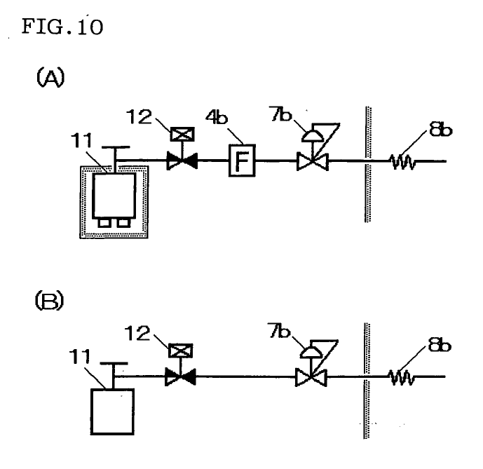

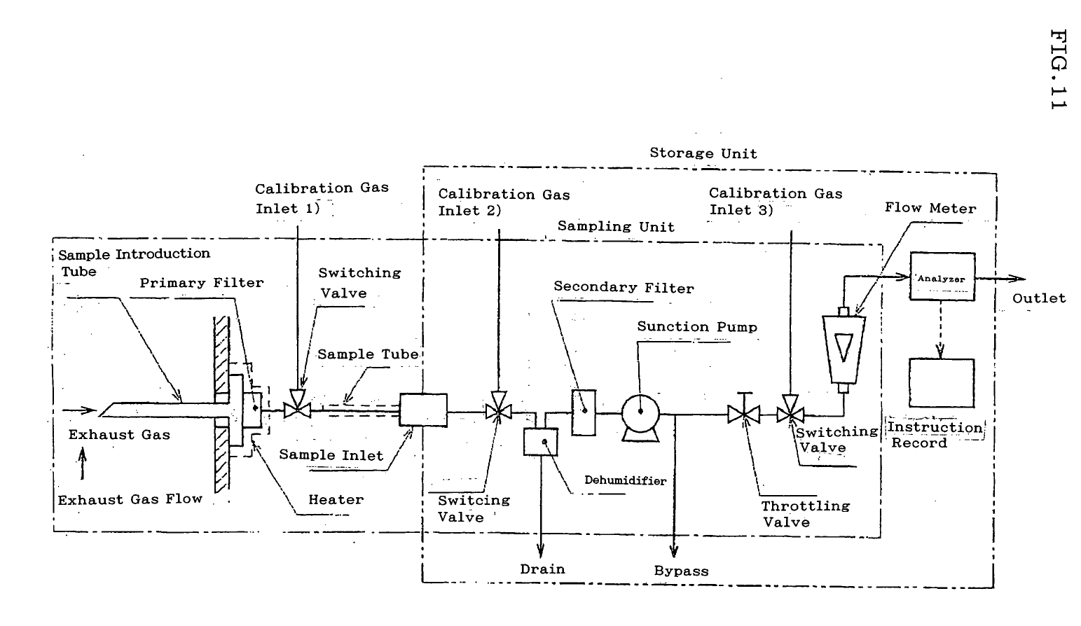

From the above results, it is revealed that when Metal Hydride fundamentally composed of the AB5-type is used, the ambient temperature of the container should be kept at 20° C. or more in order to maintain the necessary flow rate of hydrogen. As the method of satisfying such conditions, it is possible to use (1) a method of utilizing heat from a battery or heat from a detector kept at a constant temperature in the unit of the FID analyzer, or (2) a method wherein as shown in FIG. 10(A), the hydrogen feeding means 11 is arranged in a thermostatic container 16 and kept at 20° C. or more by an assistant heater 16a, whereby the minimum necessary flow rate can be secured in measurement even if the ambient temperature is reduced to 20° C. or less for example in winter.

### OTHER CONSTITUTIONAL EXAMPLE OF THE TRANSPORTABLE MEASURING APPARATUS UTILIZING A HYDROGEN FLAME ACCORDING TO THE PRESENT INVENTION (THIRD CONSTITUTIONAL EXAMPLE)

FIGS. 1 and 2 show a method of using the hydrogen feeding means, which comprises removing impurities via a stop valve 12 by filter 4b such as charcoal filter and then allowing hydrogen to flow at constant rate. It was however found that just after initiation of supply of the fuel gas, residual materials at the time of production of Metal Hydride, and hydrocarbons and sulfur compounds generated from the surface of the member constituting the hydrogen feeding means 11, are contained in a very small amount in the order of ppb or less, and after a predetermined time, these impurities are reduced to a hardly detectable level.

In the third constitutional example, this phenomenon was utilized to detect a change in the background due to hydrocarbons contained as impurities during the predetermined time after ignition of hydrogen without passing a sample gas or a calibration gas, in the arrangement of filter 4b which was not placed in the fuel feeding line as shown in FIG. 10(B). It was thereby made possible to judge whether the detector normally works or not within the warm-up time to check the working of the detector. By initiating measurement with the background in a stabilized state, it became unnecessary to arrange filter 4b essential where high-pressure gas is used in the prior art.

### INDUSTRIAL APPLICABILITY

The apparatus for measuring hydrocarbons in a sample has been described, but the same technique can also be applied to a measuring apparatus utilizing a hydrogen flame such as flame photometric detection using an FPD analyzer for measuring a sulfur compound in a sample.

What is claimed is:

1. A transportable measuring apparatus for continuously measuring the concentration of a specific component in a sample by a detector utilizing a hydrogen flame,
wherein a hydrogen feeding means having Metal Hydride as fuel gas is used, a means of refining air as a burner air is used, and a battery is used as a means of feeding power to the apparatus.

2. A transportable measuring apparatus utilizing a hydrogen flame according to claim 1, which comprises a single depressurization mechanism in a hydrogen gas line fed from the hydrogen feeding means to the detector.

3. A transportable measuring apparatus utilizing a hydrogen flame according to claim 1, wherein Metal Hydride is composed fundamentally of an AB5-type Metal Hydride.

4. A transportable measuring apparatus utilizing a hydrogen flame according to claim 2, wherein Metal Hydride is composed fundamentally of an AB5-type Metal Hydride.

5. A transportable measuring apparatus utilizing a hydrogen flame according to claim 1, wherein the top of a nozzle for forming a hydrogen flame in the detector is flat, and the diameter of the flow channel in a fuel gas-jetting part at the top of the nozzle is smaller than the diameter of the flow channel in the inside of the nozzle.

6. A transportable measuring apparatus utilizing a hydrogen flame according to claim 2, wherein the top of a nozzle for forming a hydrogen flame in the detector is flat, and the diameter of the flow channel in a fuel gas-jetting part at the top of the nozzle is smaller than the diameter of the flow channel in the inside of the nozzle.

7. A transportable measuring apparatus utilizing a hydrogen flame according to claim 3, wherein the top of a nozzle for forming a hydrogen flame in the detector is flat, and the diameter of the flow channel in a fuel gas-jetting part at the top of the nozzle is smaller than the diameter of the flow channel in the inside of the nozzle.

8. A transportable measuring apparatus utilizing a hydrogen flame according to claim 4, wherein the top of a nozzle for forming a hydrogen flame in the detector is flat, and the diameter of the flow channel in a fuel gas-jetting part at the top of the nozzle is smaller than the diameter of the flow channel in the inside of the nozzle.

9. A transportable measuring apparatus utilizing a hydrogen flame according to claim 1, wherein impurities contained in the hydrogen feeding means are detected within a warm-up time of the apparatus thereby checking the working of the detector.

10. A transportable measuring apparatus utilizing a hydrogen flame according to claim 2, wherein impurities contained in the hydrogen feeding means are detected within a warm-up time of the apparatus thereby checking the working of the detector.

11. A transportable measuring apparatus utilizing a hydrogen flame according to claim 3, wherein impurities contained in the hydrogen feeding means are detected within a warm-up time of the apparatus thereby checking the working of the detector.

12. A transportable measuring apparatus utilizing a hydrogen flame according to claim 4, wherein impurities contained in the hydrogen feeding means are detected within a warm-up time of the apparatus thereby checking the working of the detector.

13. A transportable measuring apparatus utilizing a hydrogen flame according to claim 5, wherein impurities contained in the hydrogen feeding means are detected within a warm-up time of the apparatus thereby checking the working of the detector.

14. A transportable measuring apparatus utilizing a hydrogen flame according to claim 6, wherein impurities contained in the hydrogen feeding means are detected within a warm-up time of the apparatus thereby checking the working of the detector.

15. A transportable measuring apparatus utilizing a hydrogen flame according to claim 7, wherein impurities contained in the hydrogen feeding means are detected within a warm-up time of the apparatus thereby checking the working of the detector.

16. A transportable measuring apparatus utilizing a hydrogen flame according to claim 8, wherein impurities contained in the hydrogen feeding means are detected within a warm-up time of the apparatus thereby checking the working of the detector.
